(19) United States
(12) Patent Application Publication
(10) Pub. No.: US 2017/0008818 A1
(43) Pub. Date: Jan. 12, 2017

(54) MODIFIED SUPERABSORBENT POLYMER CONTAINING A FERTILIZER

(71) Applicant: LILIZ, Chilly (FR)

(72) Inventors: Laurence HERMITTE, BOUC-BEL-AIR (FR); Cyrille VINCHON, Chilly (FR)

(73) Assignee: LILIZ, Chilly (FR)

(21) Appl. No.: 15/115,792

(22) PCT Filed: Jan. 30, 2015
(86) PCT No.: PCT/FR2015/050221

§ 371 Date: Aug. 1, 2016

(30) Foreign Application Priority Data
Jan. 30, 2014 (FR) 14/50729

(51) Int. Cl. C05G 3/04; B01J 20/26; B01J 20/22; B01J 20/32; C09K 5/16; C05C 9/00; C05G 3/00; C09K 17/32; C09K 17/22; C08K 5/21; C08J 3/205; A01C 21/00; A01G 1/00; A01G 25/00; A61F 7/02; A61F 7/10; B01J 20/24

(52) U.S. Cl. CPC C05G 3/04; B01J 20/24; B01J 20/261; B01J 20/22; B01J 20/3231; B01J 20/3208; B01J 20/321; C09K 5/16; C05C 9/00; C05G 3/0047; C09K 17/32; C09K 17/22; C08K 5/21; C08J 3/2053; A01C 21/00; A01G 1/001; A01G 25/00; A61F 7/02; A61F 7/10; C08J 2333/02; C08J 2333/26; C08J 2301/28; A61F 2007/0214

(57) ABSTRACT

A modified superabsorbent polymer (SAP) comprises a SAP matrix and urea in the form of crystals that is integrated into the SAP matrix. The SAP matrix and the urea are interpenetrated. A method for producing the modified SAP may comprise a) preparing a mixture comprising at least one urea solution and at least one SAP, b) swelling the SAP in the mixture, c) crystallizing the urea in the mixture obtained upon completion of step b), d) recovering the modified SAP in the mixture, and e) optionally, forming the modified SAP recovered at step d).

The present invention concerns the technical field of superabsorbent polymers (hereinafter abbreviated as <<SAP>>). More specifically, the present invention concerns a new SAP, called <<modified SAP>> and which is intended to agricultural applications, more particularly to the growth of plants by amendment of soils (namely the fertilization of plants, as well as the retention of water).

In the context of the present invention, by <<SAP>>, it is meant a polymer capable, in the dry state, of spontaneously absorbing at least ten times, preferably at least twenty times, still more preferably at least fifty times, and still more preferably at least one hundred times, its mass of liquid, in particular water and notably distilled water. The thus absorbed liquid is integrated into the matrix of the SAP. In other words, the SAP is characterized by high water absorption capacities, from a few tens to a few thousand times its dry mass.

By <<matrix>>, it is meant, in the context of the present invention, a network of at least one polymer, preferably a three-dimensional network which is obtained, for example, by:
cross-linking,
grafting on a support, for example a ball, a particle or a granule;
grafting on a support (for example a ball, a particle or a granule) and followed by cross-linking.

It is known to use SAPs in multiple and various fields such as hygienic products, water retainers for agricultural use, for cultivation supports, treatments of wastewaters, hydrocarbons drilling, or still in other industrial uses such as the retention of accidental chemical spillages, as well as in medical applications (for example for implants or appetite suppressants or still for compresses intended to provide cold on a portion of the body of a human subject or of an animal).

Most SAPs currently in use are:
either synthetic, for example of the polyacrylamide and/or polyacrylic type;
or natural-based, for example polysaccharides, for example cellulose derivatives.

However, the synthetic SAPs present the following drawbacks:
They are not renewable and are a bit biodegradable. As such, for agricultural applications of the SAPs, the synthesis and/or the release in the soil of the constituent monomers of these SAPs is a bit desirable for ecological considerations.
In addition, if the liquid (for example water) absorption properties of the synthetic SAPs are high, their retention properties are limited and a large portion of the absorbed water is rapidly restituted during the dehydration (or in other words during the desiccation) of the SAP.

The natural-based SAPs constitute a more ecological solution which allows overcoming the problem of biodegradability inherent to the synthetic SAPs.

However, the natural-based SAPs generally present water absorption properties which are more limited than the synthetic SAPs, and at higher costs. In addition, in the case of agricultural applications, their too rapid degradation may represent a major drawback.

Thus, it is noted from the related art that the SAPs known to date, whether they are natural-based or synthetic, are not fully satisfactory for their agricultural use as a water retainer, in order to reduce the water stress of the plants. As such, by <<water stress>>, it is meant the stress experienced by a plant placed in an environment such as the amount of water used and evapotranspired by the plant is greater than the amount that it absorbs. This stress occurs in times of droughts, but also during the increase of the salinity of the medium or during cold periods. In other words, the SAPs known to date are not fully effective when they are used to improve the water properties of a soil by retaining water and restituting this water during water stress.

Moreover, in the case of medical applications, for example when the SAPs are used as one of the constituent elements of compresses configured to provide cold when they are brought into contact with a portion of the body of a human subject or of an animal, they are generally synthetic and contained in a porous packaging. They are hydrated before their use. When drying out, the evaporation of the water contained in the SAP cools the SAP. However, the generated cold remains low and the effect of relieving pain or reducing the vascular or metabolic response is a bit significant. These compresses comprising such SAPs may sometimes be placed in the refrigerator or in the freeze prior to their use in order to accumulate more cold, which requires an organization and specific equipments for handling these compresses.

Moreover, fertilizers are chemical substances which are generally classified into the following two categories:
the organic fertilizers which may be natural-based (for example from an animal or vegetal origin) or synthetic (for example urea or urea derivatives);
the mineral fertilizers which may be synthetic or derived from natural deposits.

Fertilizers are used in agriculture, in horticulture, silviculture and for gardening activities in order to provide the plants with complements of nutrient elements, so as to improve their growth and to enhance the yield and the quality of the cultivations.

Furthermore, in the medical field, there are known crystals of salts or of substances corresponding to fertilizers which are part of the constituent elements of cooling pouches (generally known as <<instant ice packs>>), and which exploit the endothermic reaction caused by the dissolution of these crystals in the presence of water in order to generate cold on a portion of the body of a human subject or of an animal with which they are brought into contact. The mineral and ureic fertilizers are synthetic substances or derived from the exploitation of natural deposits, composed of one or more of the following elements:
basic elements such as nitrogen (N), phosphorus (P) and potassium (K);
secondary elements such as calcium (Ca), sulfur (S) and magnesium (Mg),
trace elements such as iron (Fe), manganese (Mn), molybdenum (Mo), copper (Cu), boron (B), zinc (Zn), chlorine (Cl), sodium (Na), cobalt (Co), vanadium (V) and silicon (Si).

Fertilizers are generally provided either in a solid form (for example in the form of granules, pearls, or salts) or in a liquid aqueous solution.

The most commonly used fertilizers are nitrogenous fertilizers. They consist of fertilizers containing urea, as well as urea derivatives, nitric acid salts (mainly ammonium nitrate, more rarely potassium nitrate, calcium nitrate and magnesium nitrate).

The source of nitrogen as a nutrient element of the nitrogenous fertilizer varies. It may be of ureic, ammoniacal or nitric-origin. Only the nitric form (nitrate ion NO₃⁻) can be directly assimilated by plants. The ammoniacal form (NH₄⁺) form is nitrified and oxidized into a nitrate and thus becomes assimilable. In addition, the urea has to undergo a preliminary hydrolysis, in particular by enzymes (ureases) present in soils in order to be transformed into an ammoniacal form and thus become assimilable by plants.

In runoff waters, the fertilizers are in an ionic form (nitrate NO₃⁻, ammonium NH₄⁺, sulfate SO₄²⁻, phosphate PO₄³⁻, potassium K⁺, magnesium Mg²⁺, calcium Ca²⁺) with the exception of urea, which is soluble and is in its CH₄N₂O form.

Leaching is the transport of elements such as particles, solutes, ions which compose a soil, and this under the effect of the flow of infiltration waters. Leaching drives these elements from the upper layers of the soil to the deeper layers. It may have a very negative impact on the quality of underground waters and watercourses. Indeed, leaching of nitrates is the main source of eutrophication of aquatic media and of pollution of underground and surface waters. In addition, it also induces the depletion of some nutrient elements such as nitrates, Ca²⁺ and K⁺ ions for vegetation and cultivations. This is why, in the agricultural field, this loss of nutrient elements caused by leaching of soils is taken into account by being compensated with an adequate supply of fertilizer the constituent elements of which are also subjected to leaching.

Furthermore, in the case of plants fertilization, another cause of loss of the nutrient elements in the environment is the volatilization of nitrogen in the form of ammonia gas (NH₃). This is especially the case for fertilizers containing ureic nitrogen, during the urea hydrolysis.

Thus, the form in which fertilizers, in particular nitrogenous fertilizers, are currently provided, is not fully satisfactory, because they are subjected to the leaching phenomena, as well as volatilization; which lessens their effectiveness and requires their use in a larger amount in order to satisfy the needs of plants.

Hence, it would be interesting to develop a forming of fertilizers, in particular of nitrogenous fertilizers, which would overcome these leaching and volatilization drawbacks.

The present invention aims to remedy to these plants growth difficulties inherent to the problems of performances of the SAPs when they are used as a retainer and a mean for restituting water in order to overcome the water stresses of plants and to the drawbacks known to date of leaching and of volatilization of the fertilizers.

Indeed, the inventors have developed in a quite surprising manner a modified SAP which is totally innovative and which is intended to be used in agricultural applications which consist in improving the growth of plants. More specifically, depending on its composition, the modified SAP according to the invention will improve the growth of plants according to the following two agricultural applications:
either by acting as a retainer and as a means for restituting water, in a staggered manner, to plants during water stress;
or by acting as a means for fertilizing the plants.

A first object of the present invention is a modified SAP comprising a SAP matrix into which at least one fertilizer in the form of crystals is integrated, said SAP matrix and the fertilizer being interpenetrated.

Another object of the present invention is a modified SAP which is likely to be obtained by a manufacture method which comprises the following steps of:

a) Preparing a mixture comprising at least one fertilizer solution and at least one SAP;

b) Leaving the SAP swell in said mixture;

c) Crystallizing the fertilizer contained in the mixture obtained upon completion of step b);

d) Recovering a modified SAP in the mixture;

e) Optionally, performing a step of forming the modified SAP recovered at step d).

Thus, upon completion of step c), a modified SAP according to the present invention is obtained in the mixture. Indeed, this SAP likely to be obtained by the manufacture steps as detailed hereinabove comprises fertilizer crystals within its polymeric matrix. In other words, the SAP matrix and the fertilizer are interpenetrated so as to constitute a modified SAP according to the present invention.

The modified SAP recovered upon completion of step d) is mainly characterized in that the fertilizer is in the form of crystals and in that the SAP matrix and the fertilizer are interpenetrated.

This interpenetration of the SAP matrix and of the fertilizer is obtained thanks to the steps of the method as described hereinabove, namely a dilatation (or in other words a swelling of the SAP by the fertilizer solution, followed by the crystallization of the fertilizer. This absorption of the fertilizer solution, and the crystallization that follows, result in the creation of crystals throughout the entire SAP matrix. These crystals maintain the SAP matrix swelled (or in other words dilated) and create a porosity throughout the SAP; which allows preserving a rapid hydration and an excellent solubility of the fertilizer.

Optionally, the manufacture method as detailed hereinabove comprises a step e) of forming the modified SAP obtained upon completion of step c) and recovered at step d). It may consist of a granulation (for example by atomization or implemented on a fluidized-air bed), film-coating, enrobing or any other method for solid forming of the modified SAP. With this forming step, the modified SAP according to the present invention is in a form perfectly appropriate to be used in the aforementioned two agricultural applications.

When designed to be used in the plants fertilization application, the modified SAP according to the invention presents the following advantages:
First of all, the losses through leaching of nutrient elements brought by the fertilizers are reduced. Thus, the aforementioned problems of pollution and eutrophication are limited.
In addition, the losses of nitrogen, caused by the ammonia volatilization phenomenon are also reduced, and this thanks to the maintaining of moisture of the modified SAP according to the invention. Furthermore, this reduction of volatilization may be amplified if the modified SAP according to the invention is buried in the soil in an adequate manner, for example by micro-localization, at an adjusted depth, instead of deposing it at the surface, as it is generally done with commonly-used fertilizers. The deposition of the modified SAP in the soil according to the invention will intervene mainly at the time of sowing (just before, or simultaneously), but still, it is possible to consider performing it during cultivation or anticipating it during the preparation of the soils.

When designed to be used for the retention application and as a means for restituting water, the modified SAP according to the invention presents the following advantages:
It reduces the plants water stress thanks to the water retained inside said modified SAP. The deposition of the modified SAP in the soil may be performed in a micro-localized manner and may be integrated perfectly to simplified cultivation techniques which are labor-less techniques, localized work only on the row and which are known to disturb the least the physical properties of the soil. Thanks to the deposition of modified SAP in the soil according to the invention, the water properties of the soil, in particular water retention, are improved.
Not only does the modified SAP according to the invention present water retention properties better than those of known SAPs of the related art, but it further presents the advantage of enabling optimizing the restitution of water to the plants, for example in case of water stress, namely based on the pressures which may be exerted on the modified SAP.
Besides, by its design, the modified SAP according to the invention is capable of capturing waters during rainy periods, but also restituting them (or in other words redistributing them) in the soils when the water stress is such that it endangers the growth of the cultivations. As such, it should be known that the useful reserve of a soil is generally estimated as the amount of water comprised between a 0.3 bar suction (at lower pressures, the soils are almost water-saturated, therefore the release of water by a water retainer is of no interest to the plants) and a 15 bar suction (the permanent wilting point or suction limit, the pressure beyond which the plant is no longer capable of taking out water from the soil). These “limit” pressures may vary slightly depending on the plant species.

The SAP which is used at step a) of the manufacture method may be chosen among both synthetic SAPs and natural-based SAPs.

Of course, if the SAP according to the present invention is desired to be biodegradable and less toxic, then a natural-based SAP will be chosen.

Preferably, the cross-linking rate of the SAP used at step a) is comprised between 5 and 50% by mass, preferably between 10 and 25% by mass, still more preferably between 12 and 20% by mass. The cross-linking rate is the ratio of the mass, in the dry state, of the cross-linking agent comprised in the SAP and the total mass, in the dry state, of the SAP polymer and its cross-linking agent. The cross-linking allows obtaining a solid three-dimensional matrix. Thus, this avoids disintegration of the polymer matrix during the absorption of the fertilizer solution during the step b) of SAP swelling of the manufacture method, and this while exhibiting a <<porous>> structure particularly appropriate to absorb said fertilizer solution. The cross-linking also allows for a higher remanence of the SAP in situ.

Advantageously, the SAP of step a) is chosen so that its elastic modulus is comprised between 500 Pa and 8000 Pa, preferably comprised between 1000 Pa and 5000 Pa, said elastic modulus being measured by strain scanning using a rheometer when the SAP is swelled by means of a phosphate buffer (osmolarity: 300 m Osm/kg+/−10%) with a final SAP concentration in the buffer solution of 5%.

If the SAP is a natural-based SAP, it may be obtained from at least one compound chosen in the group constituted of polysaccharides, advantageously among cellulose derivatives, alginate and glycosaminoglycans (hyaluronic acid and its salts, chondroitin sulfate, dermatan sulfate, keratan sulfate, heparin/heparan sulfate).

The SAP may be chosen among:
α-glucans such as starch, amylose and amylopectin,
β-glucans such as cellulose derivatives, galactomannans such as guaran, glucomannans such as xanthan gum, fructans, (arabino)xylans, galactans, as well as their derivatives such as carboxymethyl, alkyl, hydroxyethyl and hydroxypropyl.

Preferably, the polysaccharide has a molecular mass higher than 25 000 Da.

Preferably, the polysaccharide is a carboxyalkyl, preferably a carboxymethyl or carboxyethyl polysaccharide.

Other carboxy-alkyl polysaccharides may include ester halves obtained from cyclic anhydrides such as succinic anhydride and maleic anhydride, and from additives of maleic ester half to which sulfites are added. The carboxyalkylation degree is preferably comprised between 0 and 1.5, in particular between 0.1 and 1.0 per monosaccharide unit.

If the SAP is a synthetic SAP, it may be obtained from at least one compound chosen among polymers resulting from the polymerization with partial cross-linking of water-soluble ethylenically unsaturated monomers, and preferably in the group constituted of:
acrylic polymers, methacrylic polymers (derived in particular from the polymerization of the acrylic and/or methacrylic acid and/or acrylate and/or methacrylate monomers), vinyl polymers, in particular cross-linked and neutralized poly(meth)acrylates, in particular in the form of a gel, as well as the salts of these polymers, in particular the alkaline salts such as the sodium or potassium salts of these polymers;
polyacrylamides, and in particular in the form of a gel, as well as their salts (for example the sodium and potassium salts of these polymers);
acrylamide/acrylic acid copolymers, and in particular their salts, for example sodium or potassium salts;
polyacrylonitriles obtained by grafting on a natural or synthetic support and by chain polymerization, possibly with a possible complementary cross-linking.

In particular, the SAP used at step a) of the manufacture method may be a polymer chosen among:
cross-linked sodium or potassium polyacrylates sold under the names SALSORB CL 10, 25 SALSORB CL 20, FSA type 101, FSA type 102 (Allied Colloids); ARASORB S-310 (Arakawa Chemical); ASAP 2000, Aridall 1460 (Chemdal); KI-GEL 201-K (Siber Hegner); AQUALIC CA W3, AQUALIC CA W7, AQUALIC CA W10; (Nippon Shokuba); AQUA KEEP D 50, AQUA KEEP D 60, AQUA 30 KEEP D 65, AQUA KEEP S 30, AQUA KEEP S 35, AQUA KEEP S 45, AQUA KEEP AI MI, AQUA KEEP AI M3, AQUA KEEP HP 200, NORSOCRYL S 35, NORSOCRYL FX 007 (Arkema); AQUA KEEP 10SH-NF, AQUA KEEP J-550 (Kobo); LUQUASORB CF, LUQUASORB MA 1110, LUQUASORB MR 1600, HYSORB C3746-5 (BASF); COVAGEL (Sensient technologies), SANWET IM-5000D (Hoechst Celanese);
polyacrylamides sold under the name HYDROSORB (Hydrosorb Inc.);
acrylamide/acrylic acid copolymers in the form of a sodium or potassium salt sold under the name WATERLOCK G-400 (Grain Processing Corporation),
AQUASORB 3005 (SNF Floerger), STOCKOSORB 500, STOCKOSORB 660 (Evonik Industries), FERTISORB (Fertil), TERRA-SORB (Plant Health Care Inc.).

The SAP used at step a) of the manufacture method may be composed of natural polymers and/or of synthetic polymers, grafted or cross-linked. This may be for example the SAP ZEBA (Absorbent Technology Inc.) based on a polyacrylonitrile grafted on starch.

In one embodiment of the invention, the SAP used at step a) of the manufacture method is obtained in the following manner:
there is provided a natural-based polymer, preferably a carboxymethyl cellulose (hereinafter abbreviated as <<CMC>>) which is diluted in an aqueous medium at a basic pH (higher than 12) so that the mass concentration of said polymer is comprised between 2% and 20%.
The mixture is homogenized, preferably at a temperature comprised between 15° C. and 50° C., and this preferably for a duration comprised between about 30 minutes and about 5 hours.
This natural-based polymer is cross-linked with a cross-linking agent, for example 1,4-butanediol diglycidylether (hereinafter abbreviated as <<BDDE>>). The cross-linking degree may be comprised between 5% by mass and 50% by mass, preferably between 10% and 20%. The cross-linking is preferably carried out in a water bath, at a temperature comprised between 25° C. and 50° C., and this for a duration comprised between about one hour and 30 minutes and about four hours.
The reaction medium is neutralized by adding an acid solution.
The cross-linking reaction medium is dried until the mass concentration of the cross-linked polymer in said reaction medium is comprised between 20% and 65%, preferably between 30% and 40%. The drying may be carried out in an oven (for example at a temperature comprised between 45° C. and 50° C.), in a desiccator or by lyophilization.
A forming of the cross-linked polymer is carried out, for example by fractionation or on a fluidized-air bed.
A final drying is performed in the conditions as detailed hereinabove, until the mass concentration of the cross-linked polymer is of at least 90%. This drying step may possibly be carried out before or during the cross-linked polymer forming step.

The cross-linking of the polymer, for example of the CMC, by covalent bonds allows maintaining <<superabsorbent>> properties of said polymer over time.

Advantageously, the SAP used at step a) of the manufacture method includes hydroxyl groups, and preferably groups presenting an ionic character such as for example carboxyl groups, as well as sulfates and sulfonates.

Thanks to cross-linking, the polymer, for example the CMC, has a microporous three-dimensional matrix which is particularly appropriate to capture the fertilizer solution. Nonetheless, the three-dimensional matrix should not be too loose, namely the pores of the matrix should not be too large-sized, because said fertilizer solution then will not be retained in the polymer matrix.

At step a) of the manufacture method, the SAP is advantageously in the form of granules, preferably dry or partially-hydrated granules.

At step a) of the manufacture method, the fertilizer is selected among mineral fertilizers and organic fertilizers. Preferably, the fertilizer is selected among fertilizers with a high nitrogen content.

A fertilizer with a high nitrogen content may consist, for example, of:
a composition comprising urea in which the mass concentration of nitrogen may be up to about 46%;
a composition comprising ammonium nitrate in which the mass concentration of nitrogen may be up to about 33.5%;
a composition comprising diammonium phosphate in which the mass concentration of nitrogen may be up to about 18%;
a nitrogenous solution comprising a mixture of urea and ammonium nitrate and in which the mass concentration of nitrogen may be up to about 30%.

Preferably, it is a fertilizer which comprises at least one compound selected in the group constituted of urea, urea derivatives, nitric acid salts such as ammonium nitrate, potassium nitrate, calcium nitrate and magnesium nitrate, possibly with sulfur (for example sulfates), and ammonium chloride.

Still preferably, the fertilizer is urea or a urea derivative.

In one embodiment, the fertilizer comprises no compound which includes groups presenting an ionic character in solution and which would be likely to interfere with the liquid absorption properties of the SAP.

The fertilizer solution used at step a) of the manufacture method may be prepared by dissolving at least partially at least one fertilizer as detailed hereinabove in a solvent. Preferably, the solvent is water or a mixture of water and alcohol (in other words an alcoholic solution).

Examples of alcoholic solution consist of a mixture of water and ethanol according to a volume ratio of 90/10 or 75/25.

Preferably, the mass concentration of the fertilizer in the fertilizer solution is comprised between 50% and 100% of the fertilizer, sill more preferably between 80% and 100%. These mass concentrations are expressed as fertilizer mass divided by the mass of the solvent and the fertilizer.

If the mass concentrations of fertilizer in the fertilizer solution are lower, the risk of a peripheral crystallization of the fertilizer (therefore the non-integration of the fertilizer in the SAP matrix) is increased, which is of little interest.

More specifically, the peripheral crystallization of the fertilizer should be as low as possible, so that in the plants fertilization application, the modified SAP according to the invention could limit the aforementioned leaching problem as much as possible and so that in a water retention and restitution application, the modified SAP could restitute the water in a staggered manner.

In other words, the modified SAP according to the invention will be all the more effective for the above-described agricultural applications that a maximum of fertilizer would have been crystallized within the SAP matrix and that the peripheral crystallization of the fertilizer would be as low as possible.

Preferably, the fertilizer is completely dissolved in the fertilizer solution used at step a) of the manufacture method. The dissolution of the fertilizer in a solvent such as water or in an alcoholic solution may be performed at ambient temperature.

In one embodiment of the invention, it is possible to promote the dissolution of the fertilizer by subjecting the fertilizer solution at a temperature comprised between 30° C. and 150° C.

In a particular embodiment of the invention, the fertilizer solution used at step a) of the manufacture method according to the invention is a liquid solution of pure urea maintained at a temperature higher than the melting temperature of urea, preferably a temperature higher than 133° C. Indeed, the melting temperature of urea is of about 133° C.

In an advantageous embodiment of the invention, the fertilizer solution used at step a) of the manufacture method is an aqueous urea solution whose mass concentration of urea is of about 96% and which is obtained by dissolving urea at a temperature higher than 120° C. The small amount of water (about 4% by mass) in the fertilizer solution allows avoiding the formation of biuret (namely a compound obtained by condensation of two urea molecules and elimination of an ammonia molecule).

Of course, the dissolution of the fertilizer in a solvent such as for example water or an alcoholic solution is perfectly within the reach of those skilled in the art who, depending on the fertilizer and on the solvent he will select, will know how to dissolve the fertilizer in an adequate manner.

Advantageously, the fertilizer solution presents a slightly acid pH (namely comprised between 5 and 7, preferably between 6 and 6.5). This slight acidity of the fertilizer solution may be obtained by solubilizing the fertilizer in an adapted buffered solution. In other words, the fertilizer solution used at step a) of the manufacture method according to the invention may further comprise buffers selected in an adequate manner in order to confer a slight acidity to the fertilizer solution. This has the advantage of volatilizing less in ammonia if the fertilizer used at step a) is urea.

The fertilizer concentration in the fertilizer solution is adapted depending on the use of the modified SAP according to the invention. When the modified SAP according to the invention is intended to be used for fertilizing plants, it comprises, in percent by mass:
1 to 20%, preferably 2 to 10%, still more preferably 3 to 5% of the SAP;
80 to 99%, preferably 90 to 98%, preferably 95 to 97% of a fertilizer.

When the modified SAP according to the invention is intended to be used for retaining water and restituting it to the plants in a staggered manner, it comprises, in percent by mass:
20 to 99% of the SAP;
1 to 80% of a fertilizer.

The more the concentration of the fertilizer in the fertilizer solution is high, the less the modified SAP obtained upon completion of the crystallization step c) will present a friable peripheral crystallization not integrated into the SAP matrix. Indeed, this friable peripheral crystallization is related to the evaporation of the solvent of the SAP matrix and to the volume concentration of the SAP induced by this evaporation at the time of the crystallization step c).

As explained hereinabove, the SAP which is added in the fertilizer solution at step a) may be in a dry or partially-hydrated form. SAPs with moisture contents lower than 10% will be preferred, in order not to add too much additional water to the mixture of step a) which would increase the peripheral crystallization of the fertilizer during the crystallization step c).

Advantageously, during the swelling step b), the SAP absorbs the entire fertilizer solution, and this in order to use all the fertilizer implemented during step a) for the manufacture of the modified SAP according to the present invention—in other words so that there will be no fertilizer losses during the manufacture method of the modified SAP.

This is why, in one embodiment of the invention, the SAP may contain more water and therefore present a moisture content higher than 10% to the extent that it can absorb the entire fertilizer solution of the mixture of step a). In other terms, in order to best optimize the manufacture of the modified SAP according to the invention, the choice of the fertilizer solution and of the SAP are closely linked. The optimization of these parameters is perfectly within the reach of those skilled in the art.

At step a), the mixture is advantageously prepared by adding the SAP in the form of a powder, pearls or granules in the fertilizer solution. The average grain-size distribution of the SAP may be selected between 0.1 mm and 4 mm, depending on the desired swelling of the SAP at step b), and depending on the application and the technique of deposition in the soil of the modified SAP according to the invention which are considered.

A stirring, preferably a mechanical stirring, may be implemented during step a) of the manufacture method in order to disperse the SAP in a homogenous manner in the fertilizer solution or in other words in order to improve the exchanges between the fertilizer solution and the SAP so as to promote a homogenous penetration of the fertilizer solution in the SAP.

Advantageously, during step b), the obtained swelling ratio of the SAP is significantly lower than its maximum swelling capacity at saturation. Preferably, its swelling ratio corresponds to half its absorption capacity, still more preferably to less than quarter of its absorption capacity. Indeed, a swelling ratio lower than the maximum swelling capacity of the SAP will allow limiting the crystallization of the fertilizer at the periphery of the SAP matrix mentioned above.

Advantageously, in order to maintain the fertilizer in solution and to accelerate the swelling of the SAP, the fertilizer solution and SAP mixture is heated during step b) at a temperature similar to the solution temperature of the fertilizer, that is to say for example comprised between 30° C. and 150° C.

In one embodiment of the invention, step b) is performed at ambient temperature.

The swelling of the SAP is almost spontaneous. The duration of the SAP swelling may vary between a few tens of seconds and a few tens of minutes. This depends on the SAP which is added to the fertilizer solution and the conditions of temperature and concentration of the fertilizer.

Upon completion of step b), a radical change of the state of the mixture is observed. Indeed, the mixture comprising the fertilizer solution and the SAP which was initially liquid is henceforth in a solid form, composed of an agglomerate of SAP blocks containing in its matrix the fertilizer solution. These blocks are flexible, translucent or opalescent because they contain the fertilizer solution.

Preferably, the SAP and the fertilizer solution are selected in such a manner that upon completion of the step b) of swelling the SAP, there is no longer any supernatant which would induce a peripheral crystallization of the fertilizer out of the polymer matrix. This optimization of step b) is within the reach of those skilled in the art.

At step c), the fertilizer contained in the mixture obtained at the completion of step b) is crystallized.

During step c), the fertilizer of the fertilizer solution which is absorbed by the SAP during the swelling step b) will crystallize within the SAP matrix. In other terms, the more fertilizer solution the SAP will absorb (or in other words the more it will integrate the fertilizer solution within its matrix), the more the modified SAP recovered at step d) of the manufacture method will contain fertilizer in its matrix.

Depending on the nature of the fertilizer contained in the fertilizer solution, and depending on its concentration in said fertilizer solution, the crystallization of step c) may be carried out according to different techniques, namely:
by cooling the mixture obtained upon completion of step b);
by drying the mixture obtained upon completion of step b);
by evaporation of the solvent of the fertilizer solution of the mixture obtained upon completion of step b), as well as
by lyophilization.

In one embodiment of the invention, the crystallization of step c) is carried out by cooling by bringing the mixture obtained upon completion of step b) to a temperature lower than the solubilization temperature of the fertilizer, this solubilization temperature being dependent of the concentration of the dissolved fertilizer in the fertilizer solution.

The crystallization of step c) may be carried out at a temperature comprised between 0° C. and 100° C., and optionally implemented under stirring, preferably under mechanical stirring. The mechanical mixing allows avoiding adhesion phenomena between the granules and allows exposing the surface of each of these granules to appropriate crystallization conditions.

In another embodiment of step c), the mixture obtained upon completion of step b) is dried.

The drying may be carried out by exposition to an air flow at a temperature comprised between 30° C. and 70° C. or by baking, in a desiccator or still by lyophilization.

In another embodiment of the invention, the crystallization step c) is performed at ambient temperature.

When evaporating the solvent, the concentration of the fertilizer in the fertilizer solution integrated into the SAP matrix is increased, so that the conditions of crystallization of the fertilizer are combined thereby causing the crystallization of the fertilizer, in particular within the SAP matrix. Indeed, it is possible to evaporate the solvent until the amount of fertilizer dissolved in the residual fertilizer solution is higher than the saturation concentration.

Preferably, the crystallization of the fertilizer in the mixture obtained upon completion of step b), and therefore as explained hereinabove in particular within the

SAP matrix, is achieved since the thus modified SAP becomes in the form of hard granules. In this manner, the modified SAP may be easily recovered at step d). By <<hard granules>>, it is meant granules which resist crushing and which are appropriate to be deposited throughout a distribution chain of agricultural equipments without creating agglomerates or clogging. Modified SAP with a mass moisture content lower than 10% will be preferred.

Of course, the choice of the most appropriate crystallization technique, in other words the determination of the suitable conditions for causing the crystallization of the fertilizer in the mixture obtained upon completion of step b), is within the reach of those skilled in the art, and this depending on the fertilizer solution which will be used during step a) of the manufacture method. Indeed, the crystallization diagrams of the fertilizer-solvent systems as a function of the temperature and of the concentration of the dissolved fertilizer are perfectly known and are therefore within the reach of those skilled in the art. In this manner, those skilled in the art can determine the optimum crystallization conditions in the mixture obtained upon completion of step b).

Optionally, the modified SAP recovered at step d) is formed by a forming technique known by those skilled in the art, for example by enrobing, film-coating or granulation by atomization on a fluidized-air bed.

Possibly, additives may also be added to the granules, such as nitrification retarders or urease inhibitors, and this in order to further reinforce the reduction of leaching and volatilization in the case of an application of the modified SAP for fertilizing plants.

The present invention also concerns the use of the modified SAP according to the invention in two agricultural applications.

The 1^{st} agricultural application consists in using the modified SAP according to the invention for retaining water and restituting it to the plants in case of water stress, said restitution being spread over time.

The restitution of water by the modified SAP will be influenced by:
the matrix of the SAP used in the mixture at step a) of the manufacture method: its porosity, its cross-linking degree, the nature of its monomers, its absorption capacity, the amount and the nature of its adjuvants.
the content of fertilizer solution used at step a) of the manufacture method.

The 2^{nd} agricultural application consists in using the modified SAP according to the invention for fertilizing plants.

For these two agricultural applications, the modified SAP according to the invention is advantageously buried in the soil, preferably in combination with the implementation of strip cultivation techniques.

For this purpose, the modified SAPs according to the invention which will be formed into granules are dispersed at the surface of the soil by a superficial spreading, generally carried out with centrifugal spreaders. Afterwards, the SAP granules are buried by superficial soil-working techniques which use a scarifying tool, a disc tiller or still a chisel. The average burying depth is in the range of 2 to 10 cm below the surface of the soil.

It is to be noted that the granules can only be deposited on the soil and cannot be buried as detailed hereinabove. In this case, they will be conveyed into the soil by the infiltration of runoff waters. This embodiment is not favorable because it does not allow for an effective use of water or of the nutrients contained in the granules by the plants.

Or, preferably, the SAP granules are buried in the soil in a localized manner, generally when sowing. To do so, the SAP granules are advantageously contained in hoppers fastened over the seeder, and are then distributed in a localized manner in the proximity of or in the sowing row. In this case, the burying depth is quite similar to the one obtained with a superficial spreading, but the localization makes it mastered in better and more regular way. This technique can also allow depositing smaller amounts of the modified SAP according to the invention.

The localized burying of the modified SAP according to the invention is advantageously implemented when it combines techniques of deposition of said SAP and soil-working by simplified techniques (namely without labor and strip work, only at the level of the sowing row).

The strip-working equipments may be equipped with hoppers which distribute the granules of the modified SAP according to the invention in a localized manner in the proximity of the sowing row, preferably below the sowing row. The average burying depth is in the range of 5 to 25 cm below the surface of the soil. These techniques are particularly favorable for the cultivations called root crop cultivations of maize, beetroots, potatoes and sunflower.

The combination of the strip soil-working technique with one of the two agricultural applications of the modified SAPs according to the invention is particularly favorable.

Indeed, the preservation of the physical structure and of the natural porosity of the soil in the inter-row allows maintaining better water properties: increase of the useful water reserve and limitation of the leaching and too high percolation phenomena. When used as a water retainer, in synergy with the structure of the soil in the inter-row, the modified SAP according to the invention profits from better water dynamics which enables it to be refilled with water.

Furthermore, when the modified SAP according to the invention is used for fertilizing plants, the structure of the soil in the inter-row tends to reduce the leaching phenomenon and therefore allows for a further optimized use of the added nutrients.

When the modified SAP according to the invention is used as a water retainer, between 2 and 200 kg of the modified SAP according to the invention are deposited per hectare, preferably between 5 and 80 kg of the modified SAP according to the invention are deposited per hectare, still more preferably between 10 and 40 kg of the modified SAP according to the invention are deposited per hectare.

When the modified SAP according to the invention is used as a water retainer, between 5 and 500 kg of the modified SAP according to the invention are deposited per hectare, preferably between 10 and 250 kg of the modified SAP according to the invention are deposited per hectare.

Another object of the present invention, in the medical field, is the use of a modified SAP according to the invention such as described hereinabove as a constituent element of at least one portion of a medical device which is configured to generate cold on a portion of the body of a human subject or of an animal in order to treat it and/or relieve it.

For example, such a medical device may be used for treating and/or relieving external traumas of the body such as a sprain, a tendinitis, a wound, a hematoma, a contusion, or during invasive medical actions such as an injection, an infiltration or a surgery.

This medical device may be provided in different forms such as for example a patch, a compress or still a pouch.

This medical device has a size and a flexibility adapted to the morphology of the portion of the body of the human subject or of the animal to be treated and/or relieved.

This medical device is activated, namely it provides cold on the portion of the body of the human subject or of the animal on which it is brought into contact (or in other words on the portion of the body of the human subject or of the animal on which it has been disposed), when the modified SAP according to the invention is hydrated. Indeed, when the modified SAP according to the invention is hydrated (or in other words when it enters into contact with a substance containing a liquid, preferably an aqueous solution such as water or hydrated natural salts, for example a sodium sulfate decahydrate), the fertilizer it contains is dissolved according to an endothermic reaction which therefore produces cold.

A medical device as described hereinabove, namely including at least one of its elements being a modified SAP according to the invention, presents the following advantages:
It allows relieving more effectively the pain and reducing the vascular and metabolic responses of the portion of the body of the human subject or of the animal on which it has been set (or in other words the portion of the body on which it is brought into contact), and this because it generates a cold more intense than the cold generated by medical devices known in the related art which are also configured to provide cold on a portion of the body of a human subject or of an animal.
Cold appears instantaneously and spontaneously upon contact of the substance containing a liquid (for example water) with the modified SAP according to the invention, and this without the need for any prior freezing or handling of said medical device.

The hypertonia and/or the hyperosmolarity of the fertilizer which is dissolved and which is then in the form of a solution which is absorbed in the SAP after hydration of the latter allows cleaning and dedridement the portion of the body of the human subject or of the animal to be treated and/or relieved.

Because the SAP matrix and the fertilizer are interpenetrated in the modified SAP according to the invention, when the modified SAP according to the invention is hydrated, the fertilizer is solubilized in a perfectly homogenous manner. Thus, cold is generated and diffused in a homogenous manner within the modified

SAP according to the invention before reaching the portion of the body of the human subject or of the animal brought into contact with the medical device.

Different means perfectly within the reach of those skilled in the art may be implemented in order to hydrate the modified SAP according to the invention included in such a medical device. Advantageously, the modified SAP according to the invention is hydrated with water.

Different embodiments of the medical device which includes at least one modified SAP according to the invention may be considered. Two of these embodiments are described hereinafter.

Of course, the different embodiments of a medical device configured to provide cold on a portion of the body of a human subject or of an animal are perfectly within the reach of those skilled in the art.

In a possible embodiment of the invention, the medical device includes a porous pouch in which a modified SAP according to the invention has been disposed.

Advantageously, the pouch is made of a porous material which may be a hypoallergenic polypropylene nonwoven fabric.

When it is desired to use this medical device and therefore activating it, it is enough to bring said pouch into contact with the portion of the body of the human subject or of the animal to be treated and/or relieved. Then, a liquid (for example water) is poured on said pouch. The liquid will pass through the pores of the pouch and therefore hydrate the modified SAP according to the invention; which will also results in dissolving the fertilizer it contains and therefore initiating an endothermic reaction which generates cold which will spread up to the targeted portion of the body of the human subject or of the animal. The liquid which is poured on the pouch may be stored in pods (for example recipients such as vials or ampoules filled with the liquid).

Thus, kits comprising at least one such pouch and one pod may be provided in order to generate cold on a portion of the body of a human subject or of an animal. In another possible embodiment of the invention, the kit may comprise such a pouch and a pod (for example a recipient such as described hereinabove) containing a substance which contains a liquid (for example a hydrated natural salt such as a sodium sulfate decahydrate) so that the contact of this substance with the pouch will hydrate the modified SAP according to the invention.

In another possible embodiment of the invention, the medical device comprises a pouch, preferably a porous pouch, which presents:
at least one sealed compartment comprising a substance which contains a liquid (for example an aqueous solution such as water or still better a hydrated natural salt),
at least one modified SAP according to the invention,

said compartment is configured so that said substance containing a liquid hydrates the modified SAP according to the invention upon the activation of this medical device, namely when it is desired to generate cold on a portion of the body of a human subject or of an animal. The sealed compartment may present, for example, at least one divisible or frangible wall which is configured so that said substance containing a liquid could hydrate said modified SAP according to the invention as soon as said wall is broken.

Preferably, the substance which contains a liquid is an aqueous solution.

Advantageously, the pouch is made of a porous material which may be a hypoallergenic polypropylene nonwoven fabric.

This embodiment of the medical device has the advantage that the portion of the pouch which is in contact with the portion of the body of the human subject or of the animal remains dry, since the condensation ineluctably produced at the surface of this pouch during the hydration of the modified SAP and therefore during the endothermic reaction is immediately absorbed by the SAP comprised in said modified SAP. Thus, in comparison with the other equivalent medical devices known in the related art, this medical device configured to generate cold has the advantage of not moistening and/or soiling the targeted portion of the body of the human subject or of the animal, because the modified SAP it contains according to the invention absorbs the condensates as they are produced during the endothermic reaction because of the dissolution of the fertilizer.

When the modified SAP according to the invention is used in the medical field, the modified SAP preferably comprises a matrix of a natural-based SAP. This has the advantage that the modified SAP presents a biological compatibility.

In addition, for such a use of the modified SAP in the medical field, the fertilizer is advantageously chosen among urea and ammonium salts (preferably ammonium nitrates or chlorates).

When the modified SAP according to the invention is used for a medical application such as described hereinabove, it advantageously comprises, in percent by mass:
1 to 95%, preferably 5 to 40%, still more preferably 10 to 20% of the SAP;
5 to 99%, preferably 60 to 95%, still more preferably 80 to 90% of a fertilizer.

Preferably, the medical device is sterile, and is advantageously disposable.

### DESCRIPTION OF THE FIGURES

FIG. 1 is a graph comparing the water retention properties as a function of time (in minutes) of a 1^{st} sample of a modified SAP according to the invention (in the graph: <<1^{St} sample according to the invention>>) with those of a 1^{st} sample of a SAP of the related art (in the graph: <<1^{st} comparative sample>>).

FIG. 2 is a graph comparing the water retention properties as a function of time (in minutes) of a 2^{nd} sample of a modified SAP according to the invention (in the graph: <<2^{nd} sample according to the invention>>) with those of a 2^{nd} sample of a SAP of the related art (in the graph: <<2^{nd} comparative sample>>).

FIG. 3 is a graph comparing the water retention properties as a function of time (in minutes) of the 1^{st} sample of a SAP of the related art (in the graph: <<1^{St} comparative sample>>) with those of the 2^{nd} sample of a SAP of the related art (in the graph: <<2^{nd} comparative sample>>).

FIG. 4 is a graph comparing the water retention properties as a function of time (in minutes) of the 1^{st} sample of a modified SAP according to the invention (in the graph: <<1^{st} sample according to the invention>>) with those of the 2^{nd} sample of a modified SAP according to the invention (in the graph: <<2^{nd} sample according to the invention>>).

FIG. 5 is a photograph taken with a scanning electron microscope of a portion of urea pearl.

FIG. 6 is a photograph taken with a scanning electron microscope of a portion of granule of a synthetic SAP.

FIG. 7 is a photograph taken with a scanning electron microscope of a portion of granule of modified SAP according to a first embodiment of the invention.

FIG. 8 is a photograph taken with a scanning electron microscope of a portion of granule of a modified SAP according to a second embodiment of the invention.

FIG. 9 is a graph of the evolution of the temperature as a function of time of the content of beakers filled with water in which has been immersed either urea powder, or urea pearls, or a modified SAP according to the invention.

### DESCRIPTION OF THE PHOTOGRAPHS

FIG. 5 is a photograph taken with a scanning electron microscope at a magnification of 300 times of a portion of urea pearl which is used as a fertilizer. In said photograph, it is possible to distinguish an agglomerate of urea crystals 5.

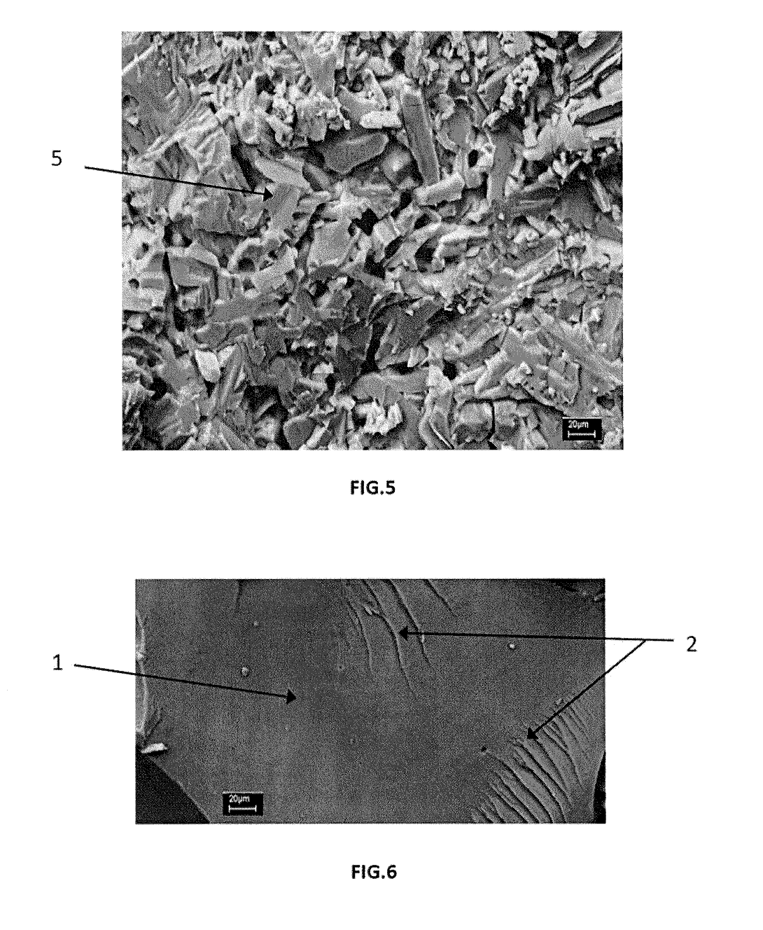

FIG. 6 is a photograph taken with a scanning electron microscope at a magnification of 300 times of a portion of granule of a synthetic SAP 1. More specifically, said SAP is a cross-linked copolymer of acrylamide and potassium acrylate which is in a non-hydrated state. In the photograph of FIG. 6, fracture lines 2 are visible. These fracture lines 2 of said SAP 1 have appeared during the mechanical fragmentation by grinding which is carried out in order to obtain the granules of the SAP 1 a portion of which is visible in the photograph of FIG. 6.

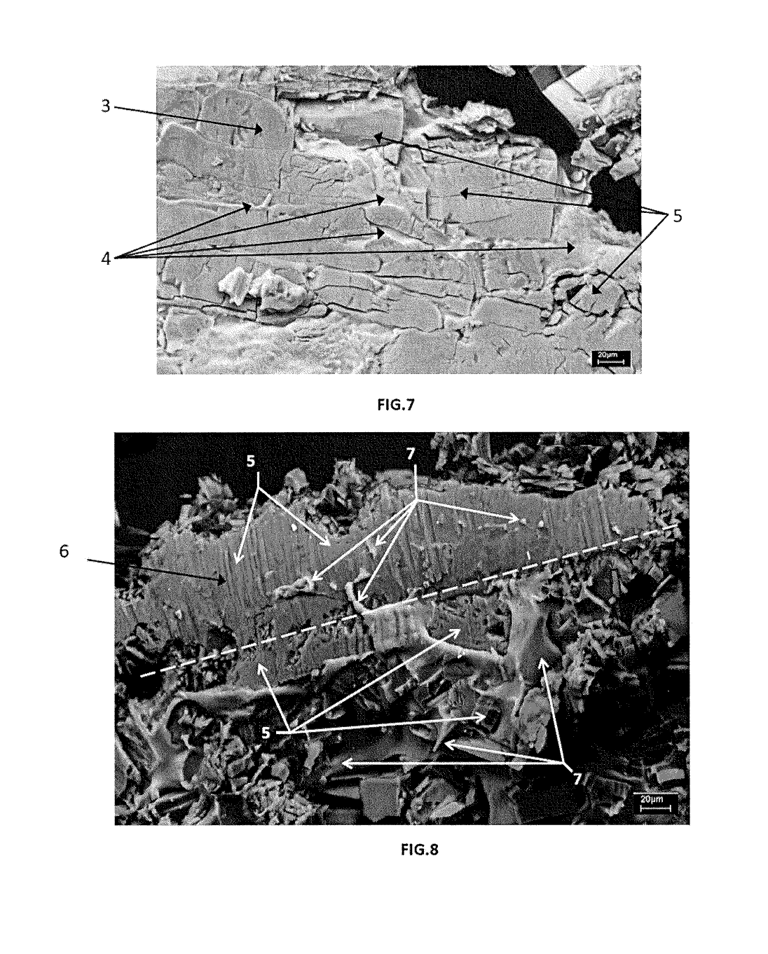

FIG. 7 is a photograph taken with a scanning electron microscope at a magnification of 300 times of a portion of granule of a modified SAP 3 according to a first embodiment of the invention. This modified SAP 3 contains, in percent by mass, 20% of a synthetic SAP of a cross-linked copolymer of acrylamide and potassium acrylate and 80% of urea. In the photograph of FIG. 7, it is possible to distinguish that the urea crystals 5 are interpenetrated with the matrix 4 of said synthetic SAP of a cross-linked copolymer of acrylamide and potassium acrylate. The matrix 4 is visible because it presents a lighter gray tone than the urea crystals 5. Thus, by comparison with the photographs of FIGS. 5 and 6 which present the starting constituents for obtaining a modified SAP according to the invention, there is noted the uniqueness of the modified SAP 3 according to the present invention which obviously presents an interpenetration of the crystals of the urea 5 with said matrix 4 of the synthetic SAP of a cross-linked copolymer of acrylamide and potassium acrylate. This interpenetration is obtained thanks to the implementation of the manufacture steps of the modified SAP 3 according to the present invention and which have been described hereinabove.

FIG. 8 is a photograph taken with a scanning electron microscope at a magnification of 300 times of a portion of granule of a modified SAP 6 according to a second embodiment of the invention. This modified SAP 6 contains, in percent by mass, 10% of a synthetic SAP of a cross-linked copolymer of acrylamide and potassium acrylate and 90% of urea. In the photograph of FIG. 8, it is possible to distinguish that the urea crystals 5 are interpenetrated with the matrix 7 of said synthetic SAP of a cross-linked copolymer of acrylamide and potassium acrylate.

In addition, the portion of the photograph of FIG. 8 which is above the dotted line is a section plane where it is possible to distinguish inclusions of said matrix 7 of the synthetic SAP of a cross-linked copolymer of acrylamide and potassium acrylate in the urea crystals 5.

Because the granule of the modified SAP 6 has been broken during cutting, this allowed highlighting, in the portion of the photograph of FIG. 8 located below the dotted line, how the matrix 7 of the synthetic SAP of a cross-linked copolymer of acrylamide and potassium acrylate is interpenetrated with the urea crystals 5 in a three-dimensional form. Hence, this photograph of FIG. 8 is very interesting, because it presents the interpenetration of the urea crystals 5 with the matrix 7 of the synthetic SAP of a cross-linked copolymer of acrylamide and potassium acrylate from different points of view, namely in section (the upper portion of the photograph) and in a three-dimensional form (the lower portion of said photograph). Thus, by comparison with FIG. 6 which also represents a synthetic SAP but within which no urea has crystallized, it is noted in the photographs of FIGS. 7 and 8 that the matrices 4, 7 of the synthetic SAPs of a cross-linked copolymer of acrylamide and potassium acrylate form a dilated network within which the urea 5 has crystallized.

Thus, the photographs of FIGS. 7 and 8 testify to the uniqueness of the modified SAP 3, 6 according to the invention in comparison with a SAP which has not been subjected to the steps of the manufacture method according to the invention which have been described hereinabove.

Experimental Part:

I—Test of the Water Retention Properties of the SAP According to the Present Invention:

The following samples are prepared:

1) 1^{st} comparative sample consisting of a SAP based on a potassium salt of a cross-linked acrylamide/acrylic acid copolymer, with an average grain-size distribution of about 0.75 mm and a mass moisture content in the range of 5% (hereinafter abbreviated as <<PAM>>). It consists of a 1^{st} comparative SAP, namely a SAP which is already known in the related art of the present invention.

2) 1^{st} sample according to the invention consisting of a modified SAP according to the invention which is obtained in the following manner:
12 g of urea are dissolved in 6 cm³ of water at a temperature of 80° C. so as to obtain a fertilizer solution.
3 g of a PAM-based SAP (namely, a SAP identical to the 1^{st} comparative sample) are added to this fertilizer solution so as to obtain a mixture;
This mixture is maintained at a temperature of 80° C., under manual stirring, until the complete integration of the fertilizer solution into the SAP which has therefore swelled. Translucent flexible small blocks of SAP, which have absorbed all the urea solution without any visible supernatant, are obtained.
The blocks are cooled for 20 minutes down to a temperature of 30° C. while maintaining a slow brewing.
The thus swelled SAP therefore containing the fertilizer is dried at a temperature of 30° C. so as to obtain a 1^{st} modified SAP according to the invention.

3) 2^{nd} comparative sample consisting of a CMC-based SAP which is obtained in the following manner:
The CMC is hydrated and linearized to 10% in a basic solution (NaOH 1% diluted in a phosphate buffer).
Said CMC solution is homogenized manually, then the mixture is subjected to a temperature of 50° C., and this for 30 minutes
A cross-linking is performed by means of a cross-linking agent consisting of a BDDE solution at 16% by mass. The cross-linking is carried out in two hours at a temperature of 50° C.
The concentration of the cross-linked CMC solution is neutralized and set to 5% by mass by adding hydrochloric acid and a phosphate buffer and the CMC matrix is left to swell, and this until obtaining a gel with a CMC concentration of 5% by mass.
A drying in a desiccators is performed at a temperature comprised 45-50° C. until obtaining a CMC concentration of 35% by mass. A SAP is thus obtained.
The thus obtained SAP is ground by means of a RAPID type 150.21 grinder with a 5 mm mesh size.
A final drying is performed. Thus, we have a 2^{nd} comparative SAP, namely according to the related art.

4) 2^{nd} sample according to the invention consisting of a CMC-based SAP which is obtained in the following manner:
12 g of urea are dissolved in 6 cm³ of water at a temperature of 80° C. so as to obtain a fertilizer solution.
3 g of CMC-based SAP (namely, a SAP identical to the 2^{nd} comparative sample obtained upon completion of the final drying) are added to this fertilizer solution so as to obtain a mixture;
This mixture is maintained at a temperature of 80° C., with manual stirring, until the complete integration of the fertilizer solution into the SAP which has therefore swelled. Opalescent flexible small blocks of SAP, which have absorbed all the urea solution without any visible supernatant, are obtained.
The blocks are cooled for 20 minutes down to a temperature of 30° C. while maintaining a slow brewing.
The thus swelled SAP therefore containing the fertilizer is dried at a temperature of 30° C. so as to obtain a 2^{nd} modified SAP according to the invention.

For each of these four samples (namely two samples according to the invention and two comparative samples corresponding to already known SAPs, or in other words according to the related art), the following steps are performed:
A total amount of 60 g of water is progressively added.
The four SAPs are left to swell, until their weight in the hydrated state is 20 times their weight in the dry state.
We waited for one hour.
3 g of each of the thus obtained four swelled SAPs are collected and placed in an oven for a desiccation at 35° C.
Measurements of the mass of each of these four SAPs are regularly performed.
The percentage of the water mass evacuated from each of the SAPs is determined by comparing the initial mass of the SAP before desiccation and its measured mass.

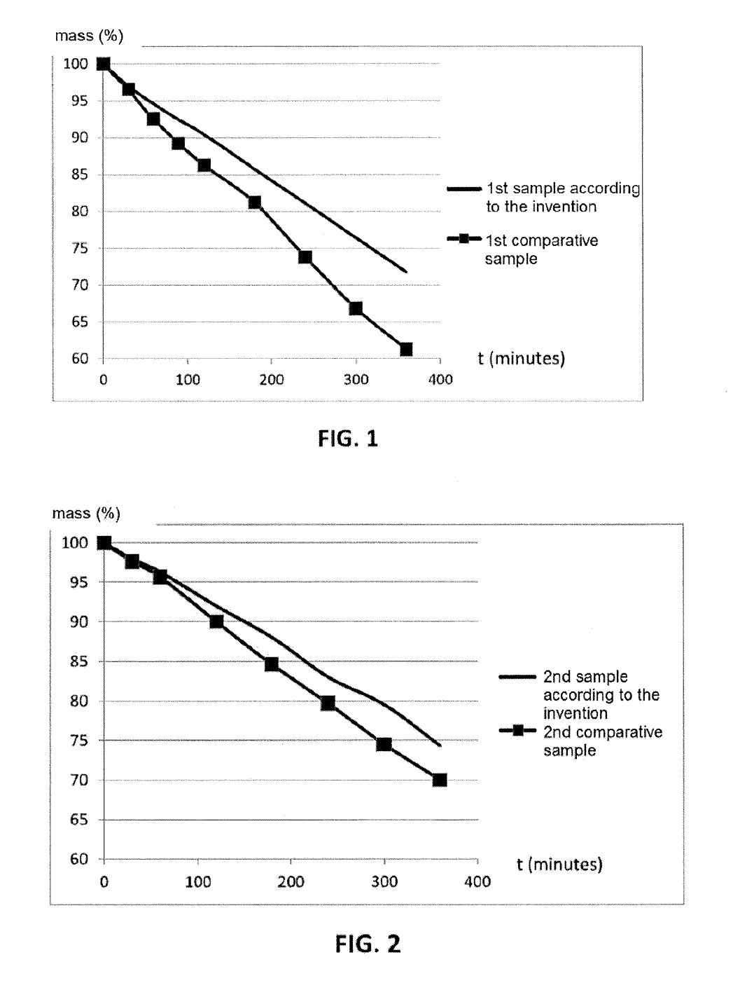

Each of the graphs of FIGS. 1 to 4 express the percentage of the water mass evacuated from the tested SAP as a function of time.

For each of the graphs, at the initial time (t=0 minutes), the initial percentage of water is 100%. The percentage of the water mass decreases over time from the initial value of 100%, in other words with the mass of water evacuated over time.

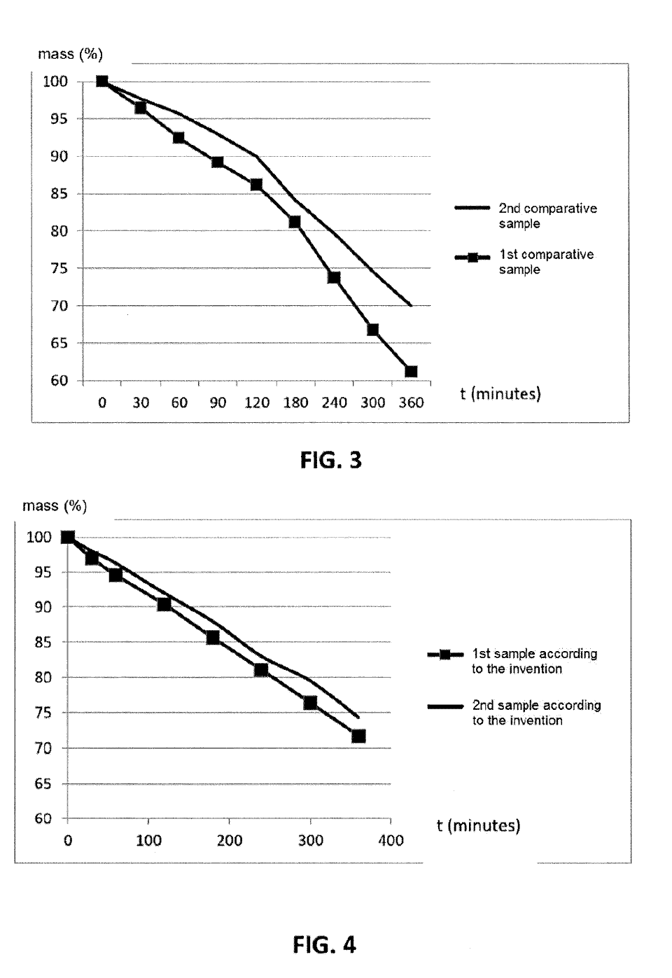

More specifically:
The graph of FIG. 1 expresses the percentage of the evacuated water as a function of time for the 1^{st} sample of modified SAP according to the invention and for the 1^{st} sample of SAP of the related art.
The graph of FIG. 2 expresses the percentage of the evacuated water as a function of time for the 2^{nd} sample of modified SAP according to the invention and for the 2^{nd} sample of SAP of the related art.
The graph of FIG. 3 expresses the percentage of the evacuated water as a function of time for the 1^{st} sample of SAP of the related art and for the 2^{nd} sample of SAP of the related art.
The graph of FIG. 4 expresses the percentage of the evacuated water as a function of time for the 1^{st} sample of modified SAP according to the invention and for the 2^{nd} sample of modified SAP according to the invention.

Given the graph of FIG. 3, it is observed that the 2^{nd} comparative SAP (namely based on the cross-linked CMC—the 2^{nd} sample of SAP of the related art) has a water retention capacity higher than that of the 1^{st} comparative SAP (namely based on the PAM—the 1^{st} sample of SAP of the related art).

It is noted in the graphs of FIGS. 1 and 2 that the SAPs according to the present invention have a better water retention capacity than their respective comparative SAPs.

Finally, it is noted that the cross-linked CMC-based modified SAP according to the invention (namely the 2^{nd} sample of modified SAP according to the invention) has a water retention capacity slightly higher than the PAM-based modified SAP according to the invention (namely the 1^{st} sample of modified SAP according to the invention).

Thus, the modified SAPs according to the invention have a fully optimized water retention capacity.

II—Test of the Plants Fertilizing Properties of the SAPs According to the Present Invention:

The following samples are prepared:

1) 1^{st} sample according to the invention consisting of a modified SAP according to the invention comprising, in percent by mass, 1% of PAM and 99% of urea which is obtained in the following manner:
12 g of urea are dissolved in 6 cm³ of water at a temperature of 80° C. so as to obtain a fertilizer solution.
120 mg of a PAM-based SAP are added to this fertilizer solution so as to obtain a mixture.
This mixture is maintained at a temperature of 80° C., under manual stirring, until the complete integration of the fertilizer solution into the SAP which has therefore swelled. Transparent flexible small blocks of SAP, which have absorbed all the urea solution without any visible supernatant, are obtained.
The blocks are cooled for 20 minutes down to a temperature of 30° C. while maintaining a slow brewing.
The thus swelled SAP therefore containing the fertilizer is dried at a temperature of 30° C. so as to obtain a 1^{st} modified SAP according to the invention in the form of quite friable granules, and presenting an urea crystallization at the surface.
The surface of the granules is removed by mechanical crushing of the urea crystals. About 4 g of friable urea crystals are thus detached from the surface and are not integrated into the modified SAP granule.

2) 2^{nd} sample according to the invention consisting of a modified SAP according to the invention comprising, in percent by mass, 5% of PAM and 95% of urea which is obtained in the following manner:
12 g of urea are dissolved in 6 cm³ of water at a temperature of 80° C. so as to obtain a fertilizer solution.
630 mg of a PAM-based SAP are added to this fertilizer solution so as to obtain a mixture.
This mixture is maintained at a temperature of 80° C., under manual stirring, until the complete integration of the fertilizer solution into the SAP which has therefore swelled. Transparent flexible small blocks of SAP which have absorbed all the urea solution without any visible supernatant, are obtained.
The blocks are cooled for 20 minutes down to a temperature of 30° C. while maintaining a slow brewing.
The thus swelled SAP therefore containing the fertilizer is dried at a temperature of 30° C. so as to obtain a 1^{st} modified SAP according to the invention in the form of granules obviously larger than the PAM-based SAP granules integrated initially to the fertilizer solution and presenting a very slight urea crystallization at the surface.
The surface of the granules is removed by mechanical crushing of the urea crystals. Less than 1 g of urea crystals is thus detached from the surface and is not integrated into the modified SAP granule. Almost all the urea is integrated (namely more than 90% of the urea) into the modified SAP granule.

3) 3^{rd} sample according to the invention consisting of a modified SAP according to the invention comprising, in percent by mass, 20% of PAM and 80% of urea which is obtained in the following manner:
12 g of urea are dissolved in 6 cm³ of water at a temperature of 80° C. so as to obtain a fertilizer solution.
3 g of a PAM-based SAP are added to this fertilizer solution so as to obtain a mixture.
This mixture is maintained at a temperature of 80° C., under manual stirring, until the complete integration of the fertilizer solution into the SAP which has therefore swelled. Transparent flexible small blocks of SAP which have absorbed all the urea solution without any visible supernatant, are obtained.
The blocks are cooled for 20 minutes down to a temperature of 30° C. while maintaining a slow brewing.
The thus swelled SAP therefore containing the fertilizer is dried at a temperature of 30° C. so as to obtain a 1^{st} modified SAP according to the invention in the form of slightly swelled granules, and presenting no urea crystallization at the surface.
No urea crystal has detached from the surface by mechanical crushing. All the urea is integrated into the modified SAP granule.

The following steps are also carried out:

1) 5 g of urea are placed on a cellulose filter with a 100 μm mesh size.

2) 6.5 cm³ of water are progressively poured in 5 minutes on said cellulose filter on which the urea are disposed.

3) We waited for one hour.

4) The cellulose filter is dried at 70° C. for 6 hours.

5) The dry residues still present on the cellulose filter are recovered and weighted.

6) The steps 2) to 5) are repeated for 2 additional cycles or until no more dry residues can be recovered on the cellulose filter.

For the 2^{nd} Sample According to the Detailed Hereinabove Invention in This Part II of the Experimental Part, the Following Steps are Carried Out:

1) 5 g of the 2^{nd} sample according to the invention are placed on a cellulose filter with a 100 μm mesh size.

2) 6.5 cm³ of water are progressively poured in 5 minutes on said cellulose filter on which the 2^{nd} sample is disposed.

3) We waited for one hour.

4) The cellulose filter is dried at 70° C. for 6 hours.

5) The dry residues still present on the cellulose filter are recovered and weighted.

6) The steps 2) to 5) are repeated for 2 additional cycles.

Table 1 below details the amounts of recovered dry residues depending on whether the test is carried out with urea or with a modified SAP according to the invention (namely a SAP containing urea—2^{nd} sample according to the invention in this part II of the experimental part).

TABLE 1
amount of recovered dry residues.

| | | |
|---|---|---|
| | | Test with the 2^{nd} |
| Amount of dry residues | | sample according to |
| recovered at step 5) upon | Test with | the invention |

| completion of the: | urea | granules | flakes |
|---|---|---|---|
| 1^{st} cycle: | 0.3 g | 4.6 g | 0.4 g |
| recovered dry residues (g) | | | |
| 2^{nd} cycle: | 0 g | 4.4 g | 0.2 g |
| 3^{rd} cycle: | not | 4.1 g | 0.3 g |
| | measured | | |

By 1^{st} cycle, it is meant the amount of dry residues recovered upon completion of step 5) carried out for the 1^{st} time.

By 2^{nd} cycle, it is meant the amount of dry residues recovered upon completion of step 5) carried out for the 2^{nd} time.

By 3^{rd} cycle, it is meant the amount of dry residues recovered upon completion of step 5) carried out for the 3^{rd} time.

The different cycles simulate the aforementioned leaching phenomenon to which the fertilizers are subjected. Each cycle also simulates the addition of water and the activation of the fertilizer dissolution endothermic reaction.

From table 1, it is noted that the urea is dissolved rapidly and has passed almost entirely through the cellulose filter. A small fraction of urea is impregnated into the filter and has crystallized at its surface.

Right from the 2^{nd} cycle, the urea residues are no longer quantifiable and all the urea is leached through the cellulose filter.

On the other hand, the modified SAP according to the invention is impregnated with water, has swelled and has absorbed all the added water.

No water drop has passed through the cellulose filter.

A small fraction of the urea contained in the modified SAP according to the invention is impregnated into the cellulose filter and has crystallized at the surface of the filter, as well as at the surface of the granules of the modified SAP according to the invention. This fraction is recovered in the form of fine flakes. This is why a column quantifying the recovered fine flakes is added in table 1 hereinabove.

Most of the urea has remained contained in the modified SAP according to the invention and only a small fraction (less than 20%) is transferred into the cellulose filter or at the surface of the granules after three consecutive cycles (in other words, leaching or activation of the endothermic reaction).

III—Test of the Solubility Properties of the Fertilizer Contained in a Modified SAP According to the Present Invention:

Another series of experiments is carried out in order to demonstrate that the fertilizer which is interpenetrated with the matrix of a SAP in order to obtain a modified SAP according to the invention preserves an excellent solubility and is retained in a dissolved form during the absorption of water by the modified SAP according to the invention.

The solubility of the fertilizer may be highlighted by the endothermic reaction which intervenes during its dissolution.

Starting from a modified SAP according to the invention which used to comprise, in percent by mass, 90% of urea crystals interpenetrated with 10% of a matrix of a synthetic SAP of an acrylamide and potassium acrylate copolymer, the following steps are carried out:

1) In a beaker, maintained at ambient temperature, 45 mL of purified water are poured then we waited for the temperature to stabilize.

2) Afterwards, 33.3 g of the modified SAP according to the invention are added in this beaker as described hereinabove (in other words, a modified SAP according to the invention which used to contain 30 g of urea).

3) The temperature of the content of the beaker is measured over time, while maintaining a slight stirring in order to homogenize and promote the dissolution of the urea contained in the modified SAP, and this as long as the content of the beaker contain two phases (solid/liquid).

In addition, two control tests are carried out by replacing the modified SAP according to the invention with two types of urea:
urea for a laboratory use (commercialized by the Sigma company) in the form of a powder with a fine grain-size distribution, namely smaller than 150 μm;
urea for an agricultural use (commercialized by the OCl-nitrogen company) in the form of pearls with a diameter of about 3 mm.

The steps 1) to 3) are also carried out on these two control tests then a record of the temperatures of the content of the two beakers, in which are added either urea powder or urea pearls, is performed.

Table 2 below details the results of the records of temperature of the contents of the beakers in which are added either the urea powder, or the urea pearls, or the urea interpenetrated with the matrix of a SAP (namely a modified SAP according to the invention).

TABLE 2
record of the temperatures as a function of time for two control
tests and for the modified SAP according to the invention

| | temperature (° C.) | | |
|---|---|---|---|
| | | | Modified SAP |
| time | Urea | Urea | according to |
| (min) | (powder) | (pearls) | the invention |
| 0 | 19.3 | 19.2 | 19.6 |
| 0.25 | 1.6 | 10.4 | 8 |
| 0.5 | 1.1 | 8 | 6.3 |
| 0.75 | 1.9 | 7.8 | 4.9 |
| 1 | 2.2 | 8.2 | 3.9 |
| 1.5 | 2 | 6.3 | 2.9 |
| 2 | 2.4 | 5.6 | 2.3 |
| 3 | 2.8 | 4.1 | 1.7 |
| 4 | 3.4 | 3.9 | 1.6 |
| 5 | 4 | 4 | 1.6 |
| 6 | 4.7 | 4.4 | 1.8 |
| 7 | 5.3 | 4.8 | 2 |
| 8 | 5.9 | 5.2 | 2.2 |
| 9 | 6.5 | 5.7 | 2.5 |
| 10 | 7 | 6.3 | 2.8 |
| 11 | 7.5 | 6.8 | 3.1 |
| 12 | 8.1 | 7.4 | 3.4 |
| 13 | 8.7 | 7.9 | 3.6 |
| 14 | 9.2 | 8.5 | 3.9 |
| 15 | 9.8 | 9 | 4.2 |

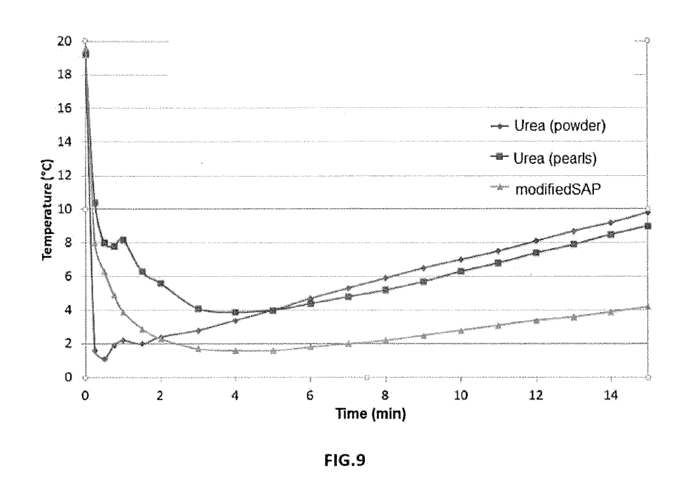

FIG. 9 is a graph of the evolution of temperature as a function of time of the content of these beakers filled with water in which is immersed either urea powder, or urea pearls, or a modified SAP according to the invention.

Given the results of table 2 and the graph of FIG. 9, it is noted that the most rapid drop in temperature is observed for the beaker which contains the urea powder, followed by the beaker containing the modified SAP according to the invention, and finally the beaker containing the urea pearls.

Considering the rapidity of the endothermic reaction, the dissolution of the urea in fine powder is therefore the most rapid.

In addition, given these results of table 2 and the graph of FIG. 9, it is noted that thanks to the porosity created in the modified SAP because of the urea crystals are interpenetrated with the SAP matrix, the absorption of water by the modified SAP according to the invention is rapid and the urea, although retained in the modified SAP, is dissolved more rapidly than the urea of the urea pearls for agricultural use.

1. A modified superabsorbent polymer (SAP) comprising a SAP matrix and urea in the form of crystals that is integrated into the SAP matrix, the SAP matrix and the urea being interpenetrated.

2. The modified SAP according to claim 1, obtained by a method that comprises:
a) preparing a mixture comprising at least one urea solution and at least one SAP;
b) swelling the SAP in the mixture;
c) crystallizing the urea in the mixture obtained upon completion of step b);
d) recovering the modified SAP in the mixture; and
e) optionally, forming the modified SAP recovered at step d).

3. The modified SAP according to claim 1, wherein the SAP is obtained from at least one compound selected from the group consisting of cellulose derivatives, alginate and glycosaminoglycans.

4. The modified SAP according to claim 1, wherein the SAP is obtained from at least one compound from the group consisting of acrylic polymers, methacrylic polymers, vinyl polymers, polyacrylamides and salts thereof, acrylamide/acrylic acid copolymers and salts thereof, and polyacrylonitriles.

5-7. (canceled)

8. The modified SAP according to claim 2, wherein the crystallization step c) is carried out by a cooling, drying, evaporation or lyophilization technique.

9. A method of using the modified SAP according to claim 1, comprising retaining water with the modified SAP and restituting the water to plants in a staggered manner, wherein the modified SAP comprises in percent by mass:
20 to 99% of the SAP; and
1 to 80% of the urea.

10. A method of using the modified SAP according to claim 1, comprising fertilizing plants with the modified SAP, wherein the modified SAP comprises in percent by mass:
1 to 20% of the SAP; and
80 to 99% of the urea.

11. A method of using the modified SAP according to claim 1, comprising burying the modified SAP in soil.

12. A medical device comprising the modified SAP according to claim 1, wherein the medical device is configured to generate cold on a portion of the body of a human subject or an animal.

13. The medical device according to claim 12, wherein the medical device is in the form of a patch, a compress or a pouch.

14. The medical device according to claim 12,
further comprising a pouch that includes at least one sealed compartment comprising at least one divisible or frangible wall and a substance that contains a liquid, wherein the compartment is configured so that the substance hydrates the modified SAP when the wall is broken.

15. The medical device according to claim 14, wherein the substance that contains the liquid is an aqueous solution.

16. The method according to claim 11, wherein the modified SAP is buried in the soil in combination with implementing strip cultivation techniques.

17. The medical device according to claim 14, wherein the pouch is porous.

18. A method of producing a modified superabsorbent polymer (SAP) comprising:
a) preparing a mixture comprising at least one urea solution and at least one SAP;
b) swelling the SAP in the mixture;
c) crystallizing the urea in the mixture obtained upon completion of step b);
d) recovering the modified SAP in the mixture; and
e) optionally, forming the modified SAP recovered at step d).

19. The method according to claim 18, wherein step e) is performed.

20. The method according to claim 18, wherein the crystallization step c) is carried out by a cooling, drying, evaporation or lyophilization technique.
